(19) United States
(12) Patent Application Publication
(10) Pub. No.: US 2014/0369977 A1
(43) Pub. Date: Dec. 18, 2014

(54) Targeting Tumor Neovasculature with Modified Chimeric Antigen Receptors

(71) Applicant: The University of Houston System, Houston, TX (US)

(72) Inventors: Xiaoliu Zhang, Houston, TX (US); Xinping Fu, Houston, TX (US)

(21) Appl. No.: 14/303,769

(22) Filed: Jun. 13, 2014

Related U.S. Application Data
(60) Provisional application No. 61/835,147, filed on Jun. 14, 2013.

(51) Int. Cl. A61K 35/14; A61K 45/06; C12N 7/00; C12N 15/86

(52) U.S. Cl. CPC A61K 35/17; C12N 15/86; A61K 45/06; C12N 7/00; C12N 2740/10041
USPC 424/93.21; 435/325; 435/456

(57) ABSTRACT

A T cell transduced with a chimeric antigen receptor can be administered to a host to kill cancer cells. The chimeric antigen receptor can include a targeting moiety with a strong binding affinity to αᵥβ₃ integrin, including but not limited to an echistatin polypeptide. The targeting moiety can also be modified to have a reduced binding affinity to α₅β₁ integrin.

### CROSS-REFERENCES TO RELATED APPLICATIONS

This application claims priority to U.S. provisional application No. 61/835,147, filed on Jun. 14, 2013, which is herein incorporated by reference in its entirety.

### GOVERNMENT SPONSORSHIP

NIH Reference Number R01CA132792

### BACKGROUND

Several promising immunotherapies are being developed to fight cancer. For example, a type of immunotherapy exploits the benefits of antigen specific T cells in cell-mediated immunity. T cells recognize their targets through T cell receptors (TCRs), which bind to antigenic peptides presented by the major histocompatibility complex (MHC) found on the surface of cancer cells. In the presence of co-stimulatory molecules, this binding results in activation of the T cell and subsequent lysing of the bound target cell (Van der Merwe P A, et al., Molecular interactions mediating T cell antigen recognition, Annu Rev. Immunol. 21:659-84 (2003)). However, most tumor associated antigens are self-proteins, and the immune system has developed a tolerance to them. Thus, one of the major challenges facing cancer immunotherapy is the difficulty in generating a sufficient number of high binding affinity tumor-specific T cells (Kammertoens T, et al., Making and circumventing tolerance to cancer, Eur. J. Immunol. 39:2345-53 (2009)).

Researchers have recently attempted to counter the immune system's tolerance to cancer cell antigens by genetically modifying T cells with a chimeric antigen receptor (CAR) via grafting, called T-CARs (Jena B, et al., Redirecting T-cell specificity by introducing a tumor specific chimeric antigen receptor, Blood 116:1035-44 (2010)). CARs are usually generated by joining a single chain antibody (scFv) to an intracellular signaling domain, usually the zeta chain of the TCR/CD3 complex. The most recent construction of CARs also contain a co-stimulatory molecule such as CD28 or 41BB that can improve effector cell survival and proliferation (Carpenito C, et al., Control of large, established tumor xenografts with genetically retargeted human T cells containing CD28 and CD137 domains, Proc. Natl. Acad. Sci. USA 106:3360-5 (2009)). For cancer therapy, T-CARs have at least three major advantages over natural T cell receptors. First, the antigen binding affinity of scFv is typically much higher than the binding moiety of most TCRs. A high affinity binding is desired for efficient T cell activation. Second, due to the nature of scFv-mediated antigen binding, T-CAR recognition is non-MHC restricted and independent of antigen processing. This widens the use of T-CARs to patients with different MHC haplotypes. Third, because T-CAR recognition is non-MHC restricted, their ability to target cancer cells is not hampered by a cancer cells' ability to down regulate MHC (an important mechanism by which tumor cells evade cancer immunotherapies).

CARs have been previously constructed with scFvs that bind to a variety of tumor-associated antigens (Davies D M, et al., Adoptive T-cell immunotherapy of cancer using chimeric antigen receptor-grafted T cells, Arch. Immunol. Ther. Exp. (Warsz) 58:165-78 (2010)). Encouraging preclinical data has prompted a series of clinical trials using adoptive transfer of T cells engrafted with these CARs for treatment of tumors having different tissue origins, including melanoma, lymphoma, neuroblastoma, and colorectal cancer (Davies D M, et al., Adoptive T-cell immunotherapy of cancer using chimeric antigen receptor-grafted T cells, Arch. Immunol. Ther. Exp. (Warsz) 58:165-78 (2010); Robbins P F, et al., Tumor regression in patients with metastatic synovial cell sarcoma and melanoma using genetically engineered lymphocytes reactive with NY-ESO-1, J. Clin. Oncol. 29:917-24 (2011); Kochenderfer J N, et al., Eradication of B-lineage cells and regression of lymphoma in a patient treated with autologous T cells genetically engineered to recognize CD19, Blood 116:4099-102 (2010); Porter D L, et al., Chimeric antigen receptor modified T cells in chronic lymphoid leukemia. N. Engl. J. Med. 365:725-33 (2011); Pule M A, et al., Virus-specific T cells engineered to coexpress tumor-specific receptors: persistence and antitumor activity in individuals with neuroblastoma, Nat. Med. 14:1264-70 (2008); Parkhurst M R, et al., T cells targeting carcinoembryonic antigen can mediate regression of metastatic colorectal cancer but induce severe transient colitis. Mol. Ther. 19:620-6 (2011)). Many of these trials have shown promising results, even complete remission of the established tumors in some cases.

Despite the impressive improvement of T-CARs over native T effector cells, there are significant drawbacks. For example, T-CARs do not actively migrate to the tumor site and they lack an active mechanism to extravasate into tumor tissue. One strategy developed to circumvent the cell migration problem included engineering T cells to express a chemokine receptor that can respond to tumor-associated chemokine milieu (Jena B, et al., Redirecting T-cell specificity by introducing a tumor specific chimeric antigen receptor, Blood 116:1035-44 (2010); Di Stasi A, et al., T lymphocytes coexpressing CCR4 and a chimeric antigen receptor targeting CD30 have improved homing and antitumor activity in a Hodgkin tumor model, Blood 113:6392-402 (2009)). However, although this strategy improved the effector cells' ability to migrate to the tumor, it did little to promote its ability to extravasate in tumor tissues.

Another therapeutic modality that needs improvement is nanoparticle-mediated drug delivery. In recent years, nanoparticles have been extensively explored as a promising delivery vehicle for chemotherapeutics. Nanoparticles have the potential to override the poor biopharmaceutical properties of many small-molecule drugs and alter their pharmacokinetics. However, despite their tremendous promise, nanoparticle-mediated antineoplastic drug delivery has been less optimal than anticipated. The preferential biodistribution and retention of nanoparticles to malignant tissues relies on the poorly organized, often leaky blood vessels and lack of lymphatics within solid tumors, a feature termed the enhanced permeability and retention (EPR) effect (Greish K, Enhanced permeability and retention (EPR) effect for anticancer nanomedicine drug targeting, Methods Mol. Biol. 624:25-37 (2010)). However, the ability of EPR to facilitate nanoparticle delivery to solid tumors remains controversial. A clearly defined mechanism that can facilitate nanoparticles to distribute to tumor tissues would greatly improve their usefulness in clinical application.

Thus, there is need in the art for a modified T cell that can overcome the barrier of blood vessel walls so that the modified T cells can get access to the tumor cells after systemic administration. There is also need in the art for methods and compositions to increase the permeability of tumor blood vessels to allow preferential deposition of nanoparticles to tumor tissues.

### SUMMARY

In an embodiment, a T cell can be engrafted with a chimeric antigen receptor that includes a targeting moiety with a strong binding affinity to αᵥβ₃ integrin. In another embodiment, the targeting moiety can be an echistatin polypeptide. In yet another embodiment, the targeting moiety can be modified to have a reduced binding affinity to α₅β₁ integrin.

In one embodiment, a T cell transduced with a chimeric antigen receptor can be administered to a host to kill cancer cells. The chimeric antigen receptor can include a targeting moiety with a strong binding affinity to αᵥβ₃ integrin, including but not limited to an echistatin polypeptide. In one embodiment, the targeting moiety can be modified to have a reduced binding affinity to α5β1 integrin.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It's understood, however, that the inventive concepts disclosed herein are not limited to the precise arrangements and instrumentalities shown in the figures.

FIG. 1A illustrates a schematic illustration of eCAR in a retroviral vector construct, in accordance with an embodiment. FIG. 1B illustrates the transduction efficiency of eCAR, in accordance with an embodiment.

FIG. 2A illustrates flow cytometry analysis of αᵥβ₃ expression on HUVEC, in accordance with an embodiment. FIG. 2B illustrates cytolysis of HUVEC by T-eCAR, in accordance with an embodiment. FIG. 2C illustrates quantification of IL-2 release during T-eCAR and T-Her2CAR mediated killing of target cells, in accordance with an embodiment. FIG. 2D quantification of IFN-γ release during T-eCAR and T-Her2CAR mediated killing of target cells, in accordance with an embodiment.

FIG. 3A illustrates flow cytometry analysis αᵥβ₃ expression on B16-F0 cells, in accordance with an embodiment. FIGS. 3B-3C illustrate cytolytic effect of T-eCAR against B16-GFPluc, in accordance with an embodiment.

FIGS. 4A-4B illustrate selective destruction of tumor blood vessels after systemic administration of T-eCAR, in accordance with an embodiment.

FIG. 5A-5B illustrate the therapeutic effect of T-eCAR against an established solid tumor of either melanoma (5A) or prostate cancer (5B), in accordance with an embodiment.

FIGS. 6A-6B illustrate that T-eCAR enhances tumor distribution of rhodamine-labeled nanoparticles following their systemic delivery, in accordance with an embodiment.

FIG. 7 illustrates that pre-administration of T-eCAR potentiates the therapeutic effect of antiangiogenic drug (AAD).

### DETAILED DESCRIPTION

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented systems, methods, features, and advantages will be or become apparent to one with skill in the art upon examining the drawings and the detailed description herein. It's intended that all such additional systems, methods, features, and advantages be protected by the accompanying claims.

All references cited in this application are incorporated in their entirety herein.

Almost all chimeric antigen receptors (CARs) are constructed to bind to tumor-associated antigens expressed on the surface of tumor cells. In an embodiment, an alternative is to make a CAR that targets tumor-associated neovasculature. This approach overcomes the inefficient tumor parenchyma penetration problems associated with other T-CARs. For example, in one embodiment, a CAR comprises an echistatin as a targeting moiety (hereafter “eCAR”). In another embodiment, the CAR can be constructed by linking a peptide sequence from echistatin to the zeta chain of a T cell. Echistatin is a 49 amino acid disintegrin that can be found in Echis carinatus venom (SEQID: 001). It has a strong binding affinity to αᵥβ₃ integrin, which is abundantly expressed on the surface of endothelial cells of tumor neovasculature (Kumar C C, et. al., Biochemical characterization of the binding of echistatin to integrin alphavbeta3 receptor, J. Pharmacol. Exp. Ther. 283:843-53 (1997); Cai W, et. al., Chen X. Anti-angiogenic cancer therapy based on integrin alphavbeta3 antagonism, Anticancer Agents Med. Chem. 6:407-28 (2006)).

In another embodiment, the selected echistatin comprises a modified DNA sequence in which the 28^{th} amino acid methionine is replaced with leucine to reduce its binding to α₅β₁ (SEQID: 002) (Wierzbicka-Patynowski I, et al., Structural requirements of echistatin for the recognition of alpha(v)beta(3) and alpha(5)beta(1) integrins, J. Biol. Chem. 274:37809-14 (1999)). It is important to avoid echistatin binding to α₅β₁ because unlike αᵥβ₃, α₅β₁ is commonly expressed in many healthy tissues.

In another embodiment, T cells are engrafted with eCAR (T-eCAR). In vitro, T-eCARS can efficiently lyse human umbilical vein endothelial cells and tumor cells that express αᵥβ₃ integrin. In yet another embodiment, systemic T-eCAR administration can lead to extensive destruction of tumor blood vessels, as judged by obvious bleeding in tumor tissues with no evidence of damage to normal tissue blood vessels. In still another embodiment, T-eCAR destruction of tumor blood vessels can significantly inhibit growth of established bulky tumors.

In an embodiment, T-eCAR co-delivered with nanoparticles in a strategically designed temporal order can dramatically increase nanoparticle deposition in tumor cells. In another embodiment, T-eCARs may be co-delivered with nanocarriers to increase their capability to selectively deliver antineoplastic drugs to tumor tissues.

CARs for Targeting Tumor Neovasculature

In an embodiment, a CAR is constructed to target tumor neovasculature. For example, in one embodiment, an echistatin sequence can be linked to the zeta chain of a T cell (T-eCAR). In another embodiment, the echistatin can be modified by substituting the 28^{th} amino acid methionine with leucine. This modification can substantially prevent T-eCAR from destroying healthy tissues. For example, the wild type echistatin has a strong binding affinity to three members of the integrin family, αᵥβ₃, α₅β₁, and α_{IIb}β₃. Both αᵥβ₃ and α_{IIb}β₃ have a narrow distribution. For example, αᵥβ₃ is mainly expressed on the surface of activated endothelial cells while α_{IIb}β₃ is expressed by platelets. However, α₅β₁ is more widely distributed (Cox D, et al., Integrins as therapeutic targets: lessons and opportunities, Nat. Rev. Drug Discov. 9:804-20 (2010)). Replacement of methionine by leucine in the modified echistatin decreases echistatin's binding affinity for α₅β₁ (Wierzbicka-Patynowski I, et al., Structural requirements of echistatin for the recognition of alpha(v)beta(3) and alpha(5)beta(1) integrins, J. Biol. Chem. 274:37809-14 (1999)). Furthermore, this modification does not significantly affect T-eCAR binding affinity to αᵥβ₃ or α_{IIb}β₃.

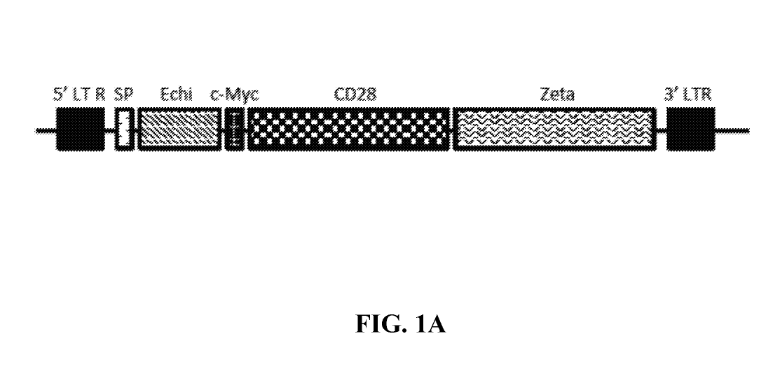

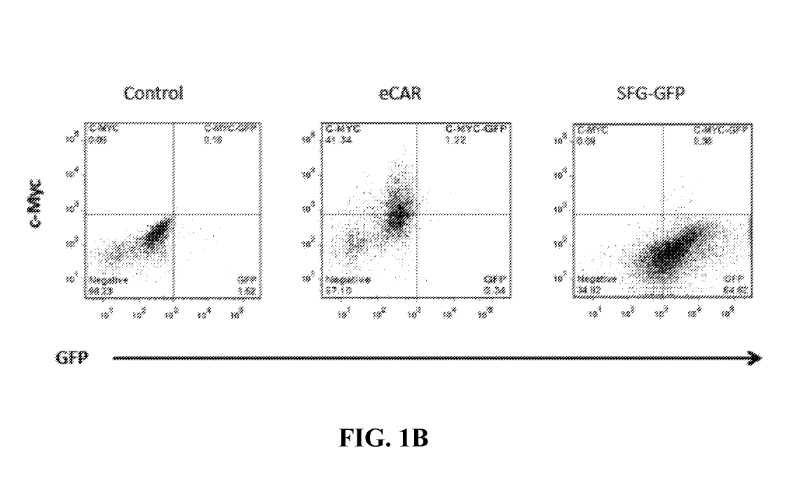

FIG. 1A, by way of example only, illustrates an embodiment of a construction of eCAR (SEQID: 003). The 5′ and 3′ long terminal repeats of the retroviral vector are labeled. The coding sequences for echistatin, CD28 (containing trans membrane domain), and zeta chain are also labeled. The DNA sequence for echistatin (Echi) can encode a modified form of echistatin. For example, the 28th amino acid, methionine, can be replaced with leucine to reduce its binding affinity to non αᵥβ₃ integrins (Wierzbicka-Patynowski I, et al., Structural requirements of echistatin for the recognition of alpha(v)beta(3) and alpha(5)beta(1) integrins, J. Biol. Chem. 274:37809-14 (1999)). The sequence may also be synthesized and inserted into a retroviral vector for stable transfection of T cells. A signal peptide (SP) may be added to the 5′ end of the fusion gene. A CD28 domain may be inserted between echistatin and zeta chain for the purpose of co-simulation function. A c-Myc tag may be inserted between echistatin and CD28 to facilitate the detection of eCAR expression. In one embodiment, the c-Myc tag is inserted during vector construction.

In an embodiment, the transduction efficiency of eCAR can be measured. Splenocytes can be transduced with either eCAR or a GFP-containing retrovirus (SFG-GFP). In an embodiment, splenocytes can be constructed by including the GFP marker gene. Mock transduced cells can be included as a negative control. The cells can be stained with PE-conjugated anti-c-Myc antibody before they are analyzed by two-color flow cytometry to detect both GFP and eCAR. Referring to FIG. 1B, by way of example only, almost half of the eCAR-transduced splenocytes are PE positive, while neither SFG-GFP-transduced nor control cells show any significant PE staining On the other hand, a high percentage of the SFG-GFP-transduced cells are GFP positive, while neither the eCAR-transduced cells nor the control cells were detectable. As a result, in one embodiment, splenocytes can be efficiently engrafted with eCAR by the retrovirus construct.

T Cells Engrafted with eCAR can Selectively and Efficiently Kill Human Umbilical Vein Endothelial Cells

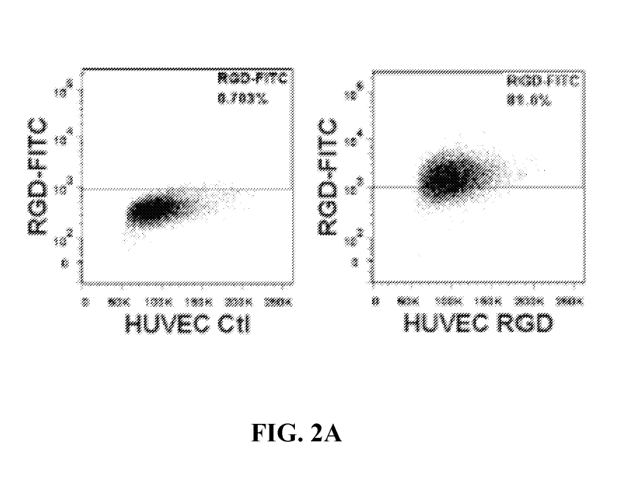

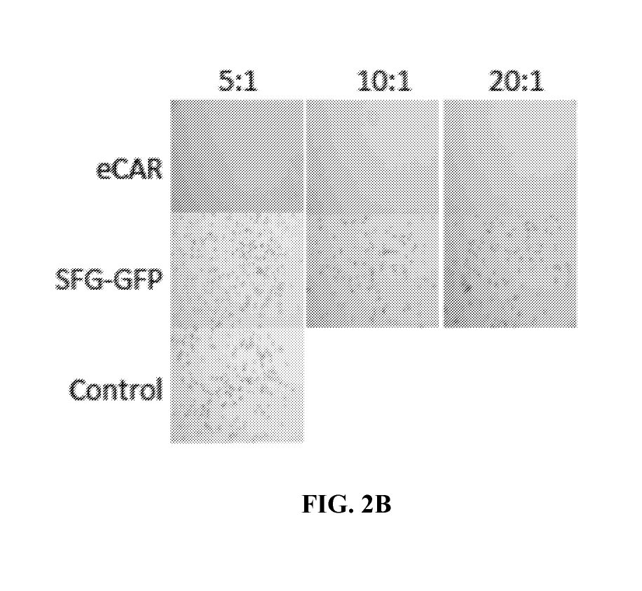

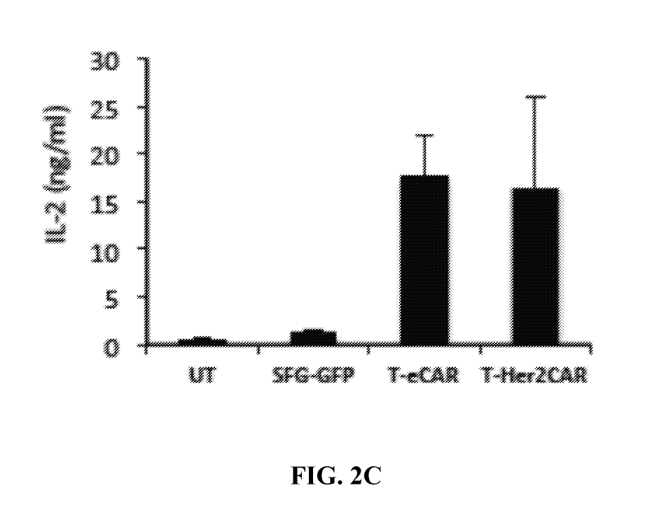

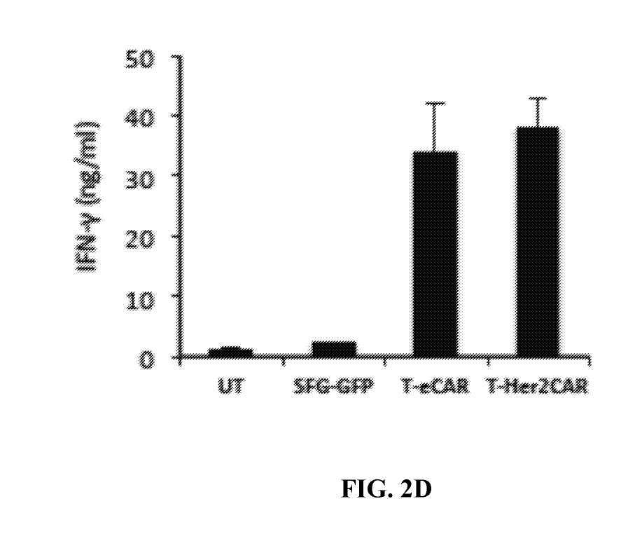

In one embodiment, to determine eCAR's effectiveness, it can be co-incubated with human umbilical vein endothelial cells (HUVEC), which express αᵥβ₃ integrin. As shown in FIG. 2A, HUVEC can be stained with FITC-conjugated RGD Peptide that also has high binding affinity to αᵥβ₃ integrin. Flow cytometry analysis shows that αᵥβ₃ integrin is highly expressed on the majority of HUVEC. In another embodiment, as illustrated in FIG. 2B, active T-eCARs and control SFG-GFP constructs can be mixed with HUVEC at different ratios for a 24 hr period to test the ability of T-eCAR to kill target cells expressing αᵥβ₃ integrin. The splenocytes can be obtained from C57BL/6 donors and transduced with retroviruses comprising either T-eCAR or a control SFG-GFP construct. T cells can then be removed by washing and the remaining monolayer can be stained with crystal violet to determine cell viability of HUVEC. A control well may represent HUVAC alone. In one embodiment, splenocytes engrafted with SFG-GFP do not exhibit any significant toxicity on HUVEC. In yet another embodiment, T-eCAR can completely lyse all the cells, even in the well with the lowest effector to target ratio.

In yet another embodiment, as illustrated in FIGS. 2C-2D, the cytokine released during T-eCAR-mediated killing of HUVEC can be measured. The results can also be compared to Her2-CAR-mediated killing of Her2-expressing tumor cells. In an embodiment, both IL-2 and interferon-γ (IFN-γ), two immune-activity signaling molecules, are released at nearly equivalent levels during cytolysis mediated by these two CARs. This indicates that the two CARs share the same killing mechanism. Furthermore, previous studies have shown that both CD4 and CD8 T cells engrafted with antigen specific TCRs can act as effector cells to efficiently kill tumor cells (Frankel T L, et al., Both CD4 and CD8 T cells mediate equally effective in vivo tumor treatment when engineered with a highly avid TCR targeting tyrosinase, J. Immunol. 184:5988-98 (2010); Kerkar S P, et al., Genetic engineering of murine CD8+ and CD4+ T cells for preclinical adoptive immunotherapy studies, J. Immunother. 34:343-52 (2011)). Therefore, in another embodiment, CD4 and CD8 T cells engrafted with eCARs can act as effector cells to efficiently kill the target cells.

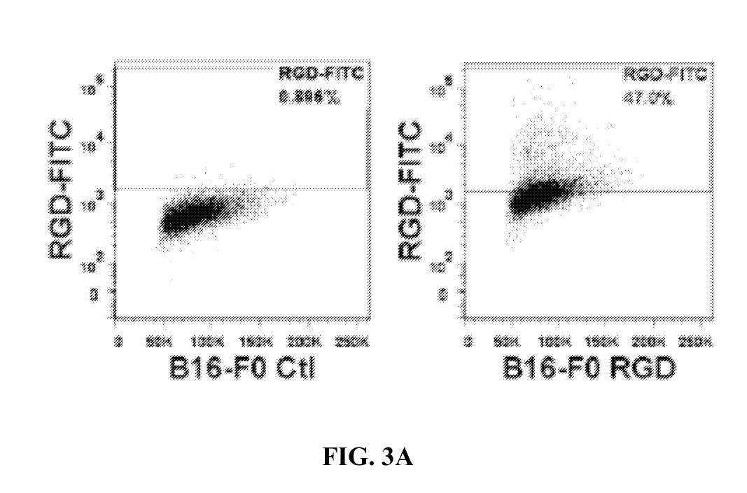

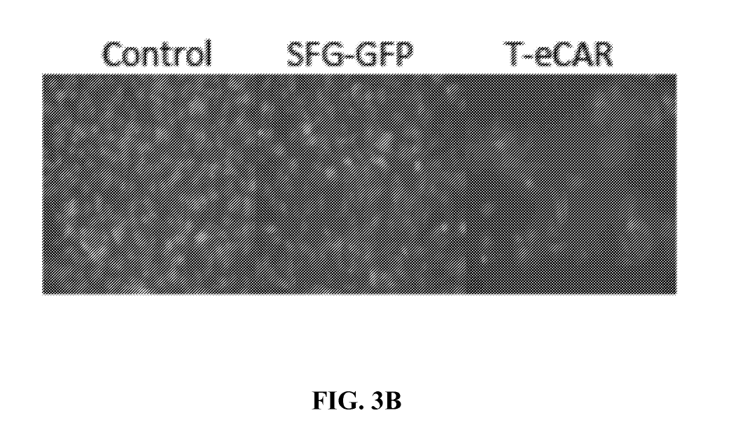

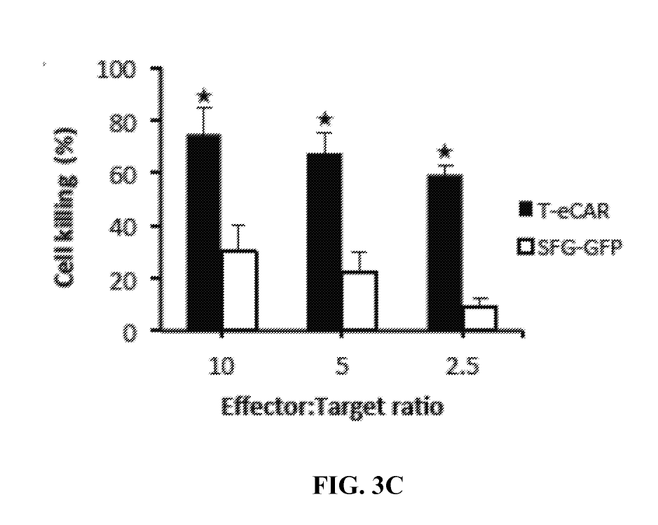

In addition to activated endothelial cells, some tumor cells that have been found to be associated with tumor metastasis have also been reported to express elevated level of αᵥβ₃ integrin (Hieken T J, et al., Beta3 integrin expression in melanoma predicts subsequent metastasis. J. Surg. Res. 63:169-73 (1996); Duan X, et al., Association of alphavbeta3 integrin expression with the metastatic potential and migratory and chemotactic ability of human osteosarcoma cells, Clin. Exp. Metastasis 21:747-53 (2004)). One tumor cell line with a higher level of αᵥβ₃ integrin expression is the B16 murine melanoma cell line (Gong W, et al., IFN-gamma withdrawal after immunotherapy potentiates B16 melanoma invasion and metastasis by intensifying tumor integrin alphavbeta3 signaling, Int. J. Cancer 123:702-8 (2008)). In one embodiment, as illustrated in FIG. 3A, the expression of αᵥβ₃ integrin in B16-F0 (parental line of B16-GFpluc) can be determined via flow cytometry after staining the cells with a FITC-conjugated RGD peptide. In an embodiment, a high percentage of B16-F0 expresses αᵥβ₃ integrin.

In yet another embodiment, as illustrated in FIGS. 3B-3C, the ability of T-eCAR to kill a B16 cell line that has been stably transduced with a fusion gene containing GFP and luciferase (B16 GFPluc) can be analyzed. For example, the cytolytic killing effect can be conveniently assessed by direct visualization of GFP (FIG. 3B). Splenocytes transduced with retroviruses containing either eCAR or the control SFG-GFP construct can be mixed with B16-GFpluc at 10:1, 5:1, and 2.5:1 ratio. The cells can be cultured for 48 hr before analysis. In an embodiment, visualization by fluorescent microscopy shows that incubation of B16-GFPluc cells with T-eCAR (E:T ratio=5:1) significantly reduces the number of GFP positive cells in the well. In yet another embodiment, visualization by fluorescent microscopy shows that splenocytes transduced with the control SFG-GFP construct show little or no effect on the integrity of the cell monolayer.

In still another embodiment, the cytolytic killing effect can be conveniently assessed by non-radioactive quantitative assay of cytolysis by measuring luciferase activity (FIG. 3C) (Fu X, et al., A simple and sensitive method for measuring tumor-specific T cell cytotoxicity. PLoS One 2010; 5:e11867 (2010)). Splenocytes transduced with retroviruses containing either eCAR or the control SFG-GFP construct can be mixed with B16-GFpluc at 10:1, 5:1, and 2.5:1 ratio. The control well can contain tumor cells only. The percentage of cell killing can be calculated by the formula: Cell killing (%)=[1−(reading of well with effector-cell)/(reading of well without effector cell)]×100. *p<0.05 as compared with SFG-GFP transduced effector cells. The cells can be cultured for 48 hr before analysis. In an embodiment, the quantitative measurement of luciferase activity shows that T-eCAR can kill at least 60% of B16-GFPluc cells even at the lowest E:T ratio (2.5:1). In another embodiment, the quantitative measurement of luceriferase activity shows that T cells engrafted with SFG-GFP construct only cause a moderate lysis of the B16 cells at the highest E:T ratio (10:1).

In an embodiment, T-eCAR has the ability to simultaneously destroy tumor neovasculature as well as tumor parenchyma if the tumor cells express elevated levels of αᵥβ₃ integrin. The methods described herein are applicable to any solid tumors that express αᵥβ₃.

In Vivo Administration of T-eCAR Induces Extensive Bleeding in Tumors but not in Normal Tissue.

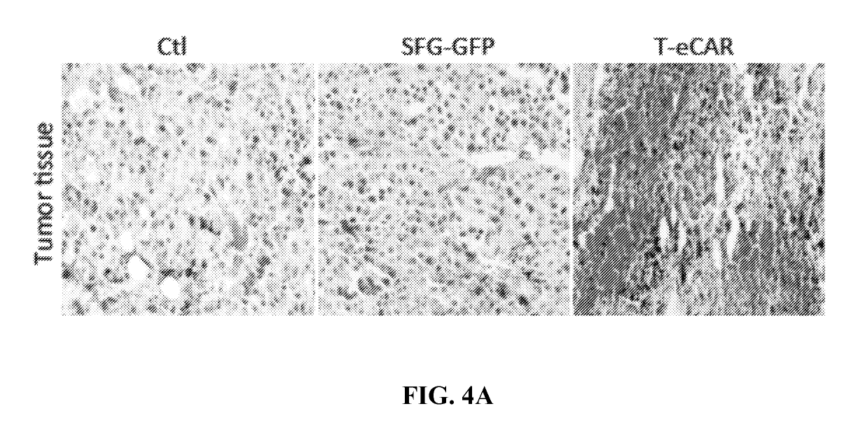

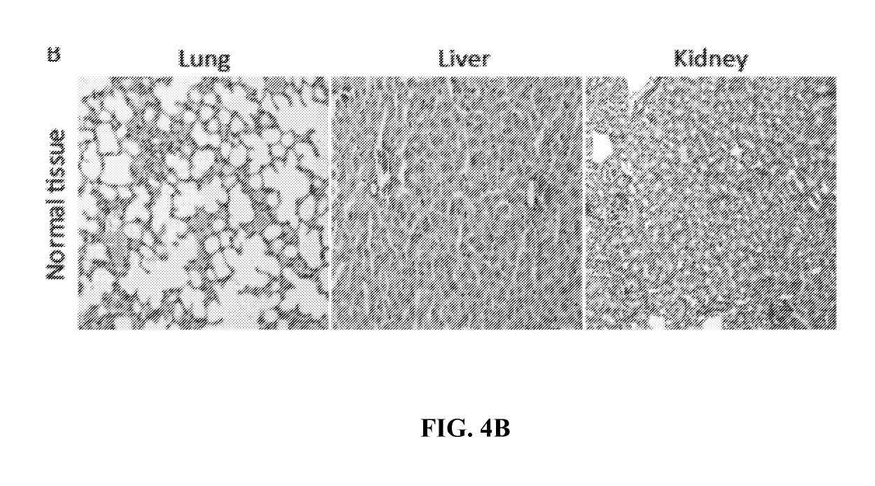

FIG. 4A, by way of example only, demonstrates an effect that T-eCAR may have on tumor blood vessels in vivo. In one embodiment, a tumor mass on the right flank of a syngeneic C57BL/6 mice can be established through subcutaneous implantation of 2×10⁵ freshly harvested B16 cells. Once the tumors reach approximately 8 mm in diameter, the mice can receive an injection (systemic infusion) via the tail vein of 5×10⁶ splenocytes transduced either with eCAR or SFG-GFP. Another group of mice may receive PBS only as a negative control. The mice can then be euthanized at day 3 after adoptive cell transfer. In an embodiment, tumors and normal organ tissues can be collected for preparation of tissue sections after paraffin embedding for histological examination and H&E staining In one embodiment, there is extensive bleeding in tumors treated with T-eCAR (FIG. 4A). Tumor parenchyma can be filled with red blood cells and other blood cell components. In another embodiment, tumors treated with SFG-GFP-transduced splenocytes exhibit very little to no bleeding. The only difference between eCAR and SFG-GFP constructs is that the latter has the echistatin coding sequence replaced by the GFP gene. Thus, in one embodiment, tumor blood vessel destruction after T-eCAR administration is primarily due to the incorporated echistatin sequence in eCAR. In yet another embodiment, tumor blood vessel destruction after T-eCAR administration is primarily due to T-eCAR's ability to bind to neovasculature-associated αᵥβ₃ integrin to trigger the T cell-mediated killing effect.

In an embodiment, normal organ tissues, including those from lung, liver and kidney, do not reveal any significant bleeding following T-eCAR administration (FIG. 4B). In another embodiment, bleeding in T-eCAR-treated tumors derives from the selective destruction of tumor vessels by the introduced T cells.

Adoptive Transfer of T-eCAR Significantly Inhibits the Growth of Established, Solid Tumors.

In one embodiment, to determine the consequence of tumor blood vessel destruction by T-eCAR, an in vivo experiment can be conducted by initially subcutaneously implanting 1×10⁵ B16 tumor cells (syngeneic murine melanoma) or PC-3 human prostate cancer cells (xenograft tumor), to the right flank of C57BL/6 mice (for B16 cells) and SCID mice (for PC-3 cells). Five days later, when the tumors become palpable, the mice can receive an intravenous systemic infusion of PBS, or 4×10⁶ splenocytes transduced with either eCAR or SFG-GFP. Mice in the third group can be given only PBS. The tumors can be measured weekly to determine tumor volume. *p<0.05, ⁺p<0.01 as compared with SFG-GFP and PBS.

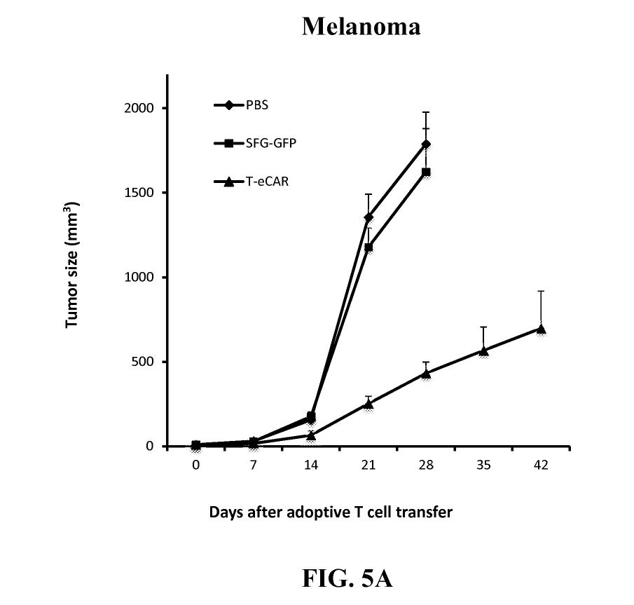

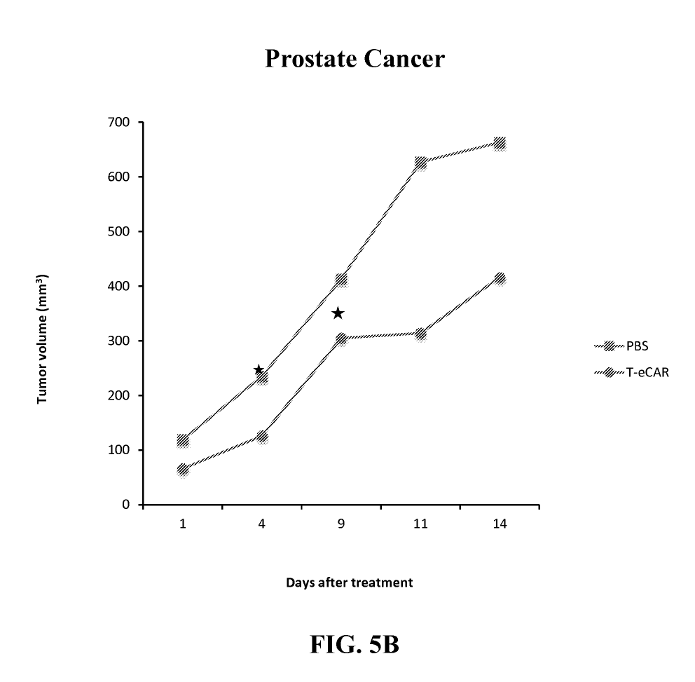

In an embodiment, as illustrated in FIG. 5A, tumor growth can be essentially unhalted in mice treated with PBS or splenocytes transduced with SGF-GFP. By the 28^{th} day following the start of treatment of B16 melanoma, the tumors in both groups can reach a large size and the animals may need to be euthanized due to reaching the preset endpoint. In contrast, in another embodiment, administering T-eCAR can effectively slow down tumor growth. By the 42^{nd} day following the start of treatment, the tumors in the T-eCAR-treated group can be relatively small and most of the animals may still be alive. In another embodiment in FIG. 5B, T-eCAR treatment led to a significantly smaller size tumor at days 11 and 14 after treatment. Thus, in both embodiments, T-eCAR-mediated destruction of tumor blood vessels can lead to significant therapeutic benefit against established solid tumors of different tissue origins.

In the embodiment in FIG. 5A, the adoptively transferred T-eCAR can attack both tumor blood vessels and the tumor cells. As such, the initial tumor blood vessel destruction can allow the efficient infiltration of the T-eCAR to tumor parenchyma to be in proximate contact with tumor cells. This can avoid the need for active extravasation, a characteristic that T-eCAR otherwise may not possess. Therefore, in still another embodiment, the combination of blood vessel destruction and the subsequent T-eCAR infiltration to directly kill tumor cells can synergize for a better therapeutic effect than either action alone.

In an embodiment, the effect of T-eCAR is maximized by combining it with antiangiogenic agents, such as angiopoietin 2, angiostatin, endostatin, platelet factor-4, avastin, aflibercept, sorafenib, sunitinib, pazopanib, vandetanib, vatalanib, cediranib, axitinib, which can prevent new tumor blood vessel formation following T-eCAR administration. In another embodiment, such a combination may produce a synergistic effect, as T-eCAR mediated tumor blood vessel destruction can convert the relatively slow process of tumor angiogenesis into an acute event that can maximize the therapeutic response to antiangiogenic compounds.

Tumor Blood Vessel Destruction by T-eCAR can Increase Nanoparticle Tumor Penetration.

In another embodiment, as illustrated in FIG. 4A, there can be significant bleeding observed in malignant tissues after infusion of T-eCAR. This indicates that T-eCAR can be used as a means to enhance nanoparticle delivery to tumors following their systemic administration. Thus, in an embodiment, T-eCAR can be administered alongside nanoparticles to increase nanoparticle permeability of tumors. These methods are applicable to all tumor blood vessels, since all tumor blood vessels contain substantially high levels of αᵥβ₃ integrin and are, therefore, sensitive to the effect of T-eCAR.

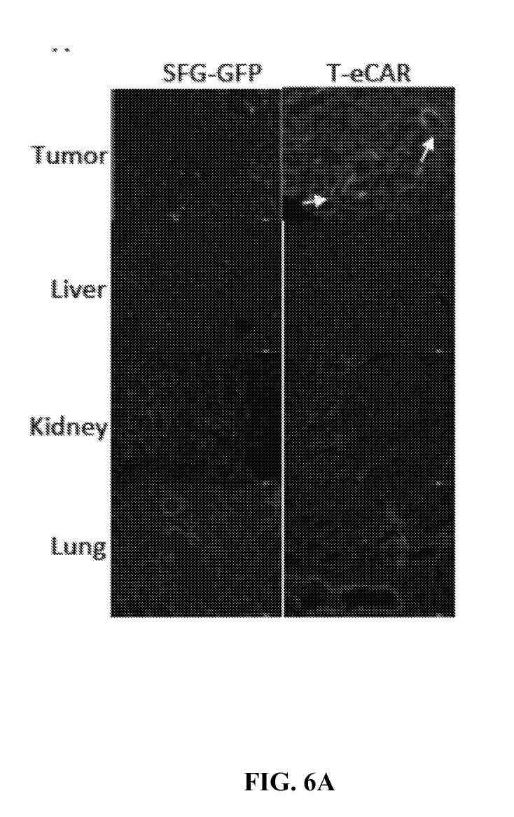

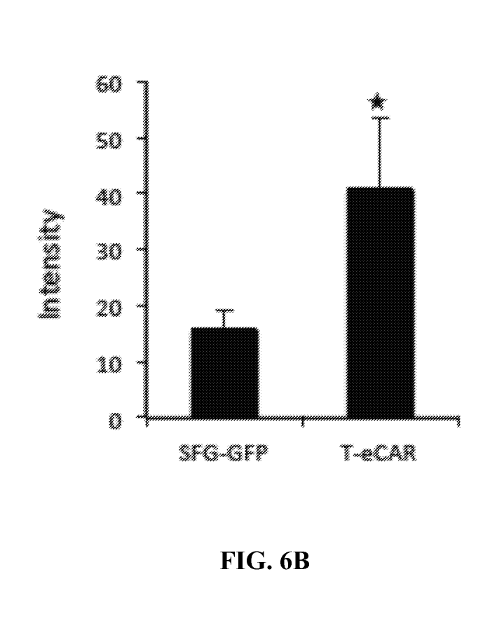

In yet another embodiment, T-eCAR or T cells transduced with the SFG-GFP control construct can be administered to mice (FIGS. 4A-4B). For example, in an embodiment, murine melanoma can be established at the right flank of C57BL/6 mice. Once the tumors reach approximately 8 mm in diameter, the mice can receive intravenous infusion of 4×10⁶ T-eCAR or SFG-GFP-transduced splenocytes. After 48 h, 10 mg/kg rhodamine-labeled liposome nanoparticles can be administered to the mice intravenously. Animals can then be euthanized one to two days after the liposome nanoparticle administration. In an embodiment, tumors and major organs can be collected to prepare frozen sections that can be used for cryo-fluorescent microscopic examination as illustrated in FIG. 6A. In one embodiment, rhodamine staining may only be sparsely seen across the tissue sections prepared from mice receiving SFG-GFP transduced T cells. In contrast, in another embodiment, widespread rhodamine staining may be seen across the entire tumor parenchyma from mice receiving T-eCAR. Visible blood vessels seem to have broken areas (indicated by white arrows). In yet another embodiment, as illustrated in FIG. 6B, quantification of rhodamine in tumor tissues confirms that there can be a significant difference between SFG-GFP and T-eCAR treatment. The tumor tissues can be quantitated using the MicroSuite™ FIVE software. Results, given by the software as value intensity, represent the mean value of 15 randomly chose areas across three slides, one from each tumor sample. *p<0.01, as compared with SFG-GFP.

Examination of tissue sections from normal organs reveals very little evidence of rhodamine deposition except in the lung, where blood vessels can be seen to be lightly stained with rhodamine. This observation is consistent with early reports that liposome nanoparticles have the tendency to get trapped in the lung after systemic delivery (Liu Y, et al., Factors influencing the efficiency of cationic liposome-mediated intravenous gene delivery, Nat. Biotechnol. 15:167-73 (1997)). Despite this mechanic trap in the lung, there is little evidence for parenchyma distribution of nanoparticles in the lung. This fact, in combination with the failure to detect any significant bleeding in the lung (and other normal organ tissues) (FIG. 4B), supports this assumption. Together these results suggest that: 1) despite the reported presence of enhanced permeability and retention (EPR) effect in tumor tissues, it does not allow efficient deposition of nanoparticles to enter into tumor interstitium after their systemic delivery, 2) T-eCAR does not cause significant damage to blood vessels in normal organ tissues despite its potent destructive effect on tumor neovasculature, and 3) tumor blood vessel destruction mediated by T-eCAR allows systemically delivered nanoparticles to efficiently enter deep into tumor parenchyma. Therefore, in an embodiment, T-eCAR can be combined with the nanoparticle-mediated drug delivery of many antineoplastic small molecules, such as 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, Thioguanine, mechlorethamine (nitrogen mustard), chlorambucil, cyclophosphamide (Cytoxan®), ifosfamide, melphalan, streptozocin, carmustine (BCNU), lomustine, busulfan, dacarbazine (DTIC), temozolomide (Temodar®), thiotepa, altretamine, cisplatin, carboplatin, oxalaplatin, Daunorubicin, Doxorubicin (Adriamycin®), Epirubicin, Idarubicin, paclitaxel (Taxol®), docetaxel (Taxotere®), ixabepilone (Ixempra®), vinblastine (Velban®), vincristine (Oncovin®), vinorelbine (Navelbine®), Estramustine (Emcyt®).

Furthermore, in an embodiment, examination of major organs after delivery of rhodamine-labeled nanoparticles does not reveal any significant blood vessel leakage. This observation can be made in the same animals that show significant damage to tumor blood vessels from T-eCAR administration. This, in combination with a failure to detect any significant bleeding in the lung and other normal organ tissues, suggests that T-eCAR is not significantly toxic to normal tissue. In another embodiment, although some endothelial cells of normal tissue express αᵥβ₃ integrin just like cancer blood cells, the level of expression is below the threshold that is readily detectable by T-eCAR.

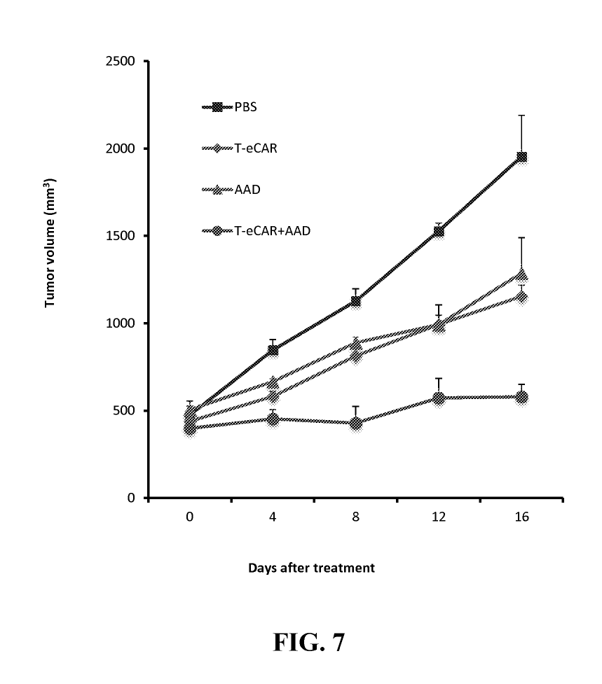

In a further embodiment, T-eCAR can be co-administered with any antiangiogenic drug (AAD) to enhance the therapeutic effect of the latter. Antiangiogenic drugs may include but are not limited to angiopoietin 2, angiostatin, endostatin, platelet factor-4, avastin, aflibercept, sorafenib, sunitinib, pazopanib, vandetanib, vatalanib, cediranib, axitinib, etc. Antiangiogenic therapy is based on a solid proposition that angiogenesis is an essential manifestation of solid tumors. Several selective antiangiogenic drugs (AADs) have been developed in recent years. However, the cadre of these compounds so far only produced largely modest effects and none of them show any improvement on overall survival. One of the main reasons for the less optimal therapeutic outcome of AAD is because the tumor blood vessel formation is a relatively slow process. This requires a prolonged treatment, during which tumors frequently develop resistance to the therapy. A combination of T-eCAR with AAD can resolve this issue. For example, in one embodiment, the initial destruction of tumor blood vessels by T-eCAR can convert the relatively slow process of tumor angiogenesis into an acute event, which will increase the responsiveness of tumor to antiangiogenic therapy. As another example, in an embodiment and as illustrated in FIG. 7, pre-administration of T-eCAR to tumor-bearing animals has dramatically enhanced the therapeutic effect of an AAD (e.g, Pazopanib). In one embodiment, colon cancer can be established on the right flank of Balb/c mice by implanting CT26 tumor cells. When tumors reach nearly 5 mm in diameter, tumors can be treated with: 1) PBS, 2) T-eCAR alone, 3) AAD alone, and 4) T-eCAR plus AAD. In another embodiment, the best therapeutic result can be obtained from the combinatorial treatment of tumors with T-eCAR plus AAD, indicating that T-eCAR mediated tumor blood vessel destruction potentiates the therapeutic effect of AAD. The therapeutic effect of any AAD can be potentiated by T-eCAR to potentiate therapeutic effect, since all AADs inhibit angiogenesis.

It's understood that the above description is intended to be illustrative, and not restrictive. The material has been presented to enable any person skilled in the art to make and use the inventive concepts described herein, and is provided in the context of particular embodiments, variations of which will be readily apparent to those skilled in the art (e.g., some of the disclosed embodiments may be used in combination with each other). Many other embodiments will be apparent to those of skill in the art upon reviewing the above description. The scope of the invention therefore should be determined with reference to the appended claims, along with the full scope of equivalents to which such claims are entitled. In the appended claims, the terms “including” and “in which” are used as the plain-English equivalents of the respective terms “comprising” and “wherein.”

### EXAMPLES

Cell lines. Human umbilical vein endothelial cells (HUVEC) and the murine melanoma cell line B16-F0 were obtained from ATCC (Manassas, Va.). HUVEC were cultured in ATCC formulated Dulbecco's Modified Eagle's Medium (DMEM; Catalog No. 30-2002) with 20% fetal bovine serum (FBS) and B16-F0 cells were grown in 10% FBS DMEM with 100 μg/ml streptomycin and 100 U/ml penicillin. B16-GFPluc cells were established in our lab by co-transfecting pIR-eGFP-luc and pCMV-piggyBac plasmids into B16-F0 followed by flow cytometry sorting and single cell cloning as previously described (Fu X, et al., A simple and sensitive method for measuring tumor-specific T cell cytotoxicity. PLoS One 2010; 5:e11867 (2010)).

Retroviral vector construction and production. The construction of retroviral vectors is schematically presented in FIG. 1A. The coding sequences for Leu-28-echistatin (MECESGPCCRNCKFLKEGTICKRARGDDLDDYCNG KTCDCPRNPHKGPAT; GenBank: M27213.1) and Myc-tag (EQKLISEEDL) were synthetized by IDT (Integrated DNA Technologies, Coralville, Iowa) containing the restriction sites XhoI and NcoI. This construct was then cloned into the vector SFG6 FRGS-CD28-Zeta, by replacing the HER2 ScFv coding sequence (Ahmed N, et al., Regression of experimental medulloblastoma following transfer of HER2-specific T cells, Cancer Res. 67:5957-64 (2007)). A signal peptide (SP) has been added to the 5′ of the fusion gene. Additionally, a c-Myc tag (c-Myc) has been inserted between echistatin and CD28 to facilitate the detection of eCAR expression. The construct was named eCAR. For constructing SFG-GFP, the GFP gene minus the stop codon was similarly inserted into SFG-FRG5-CD28-Zeta. To prepare viral stocks, the retroviral vector constructs were transfected into the retrovirus packaging cell line Platinum-E, using the FuGENE® 6 transfection reagent (Roche Applied Science Indianapolis, Ind.) (Morita S, et al., Plat-E: an efficient and stable system for transient packaging of retroviruses. Gene Ther 7:1063-6 (2000)). Supernatants were harvested 48 and 72 h later and filtered through 0.45 μm filters. The purified supernatants were combined and titrated on 293 cells to determine the virus yield.

Transduction of murine splenocytes with retroviral vectors. Splenocytes were harvested from C57BL/6 mice and cultured with RPMI 1640 medium supplemented with 25 mM HEPES, 200 nM L-glutamine, 10% FBS, 1% MEM nonessential amino acids, 1 mM sodium pyruvate, 50 μM β-mercaptoethanol, 100 μg/ml streptomycin and 100 U/ml penicillin. Cells in suspension (2×10⁶/ml) were stimulated with concanavalin A (2 μg/ml; Sigma, St. Louis, Mo.) and murine IL-2 (1 ng/ml; ProSpec, East Brunswick, N.J.) for 24 h before they were transferred to RetroNectin (Takara Bio. Inc., Shiga, Japan) coated non-tissue culture 24-well plates for transduction with eCAR or SFG-GFP retroviruses. The transduced splenocytes were then cultured for 48 hours in fresh medium supplemented with 10 ng/ml of murine IL-2.

Flow cytometry analysis for eCAR and GFP expression. Splenocytes transduced with eCAR retrovirus were washed once with PBS containing 2% fetal bovine serum before they were incubated for 30 min at 4° C. with Mouse BD Fc Block (BD Biosciences, San Jose, Calif.) that contains rat anti-mouse CD16/CD32 antibody. After washing with PBS twice, cells were stained with PE-conjugated Myc-tag mouse antibody (Cell signaling, Danvers, Mass.) or isotype antibody for 30 min at 4° C. in dark. The cells were washed twice before used for analysis. SFG-GFP transduced cells were used directly for analysis without any staining. Both cell preparations were then analyzed on BD FACSAria™ II (BD Biosciences, San Jose, Calif.), with data analysis on >10,000 events. For determining αvβ3 integrin expression, HUVEC or B16-F0 cells were stained with 10 μg of fluorescein isothiocyanate (FITC)—conjugated Arginine-Glycine-Aspartic Acid (RGD) Peptide (AnaSpec, Fremont, Calif.) in 100 μl 1% FBS-PBS for 30 min at 4° C. After washed 3 times with PBS, cells were analyzed with the same BD FACSAria™ II.

Cytotoxicity assay of retrovirus transduced splenocytes. The cytotoxicity of the retrovirus-transduced splenocytes on target cells was assayed by either visualization or by a recently reported nonradioactive quantitative measurement (Fu X, et al., A simple and sensitive method for measuring tumor-specific T cell cytotoxicity. PLoS One 2010; 5:e11867 (2010). For the visualization detection, 5×10⁴ target cells well were initially seeded to 48-well plates. Retrovirus-transduced splenocytes (effector cells) were added 24 h later at effector to target (E:T) ratios ranging from 20:1 to 2.5:1. Cells were fixed 24 or 48 h later and stained with 1% crystal violet in 20% ethanol for visualization and imaging under a light microscope. For quantitative measurement of cytotoxicity of the retrovirus-transduced splenocytes, 1×10⁴ target cells were seeded on 96 well plates first. Effector cells were added 24 h later at E:T ratios ranging from 20:1 to 2.5:1. Forty-eight h later, media was removed and cells were rinsed with PBS. Then, 50 μl of the Bright-Glo™ (Luciferase Assay System, Promega, Madison, Wis.) was added to each well. Plates were gently shaken for 2 minutes for the cells to be completely lysed. The cell lysates were then transferred into 96-well opaque plates for luminescence measurements with SpectraMax® multi-mode microplate reader (Molecular Devices, Sunnyvale, Calif.). Cytotoxic activity was calculated by the formula: Cell killing (%)=[1−(reading of well with effector-cell)/(reading of well without effector cell)]×100.

Measurement of cytokine release. Splenocytes were obtained from C57BL/6 donors. They were either untransduced (UT), or transduced with SFG-GFP, eCAR (T-eCAR) or Her2CAR (T-Her2CAR). The details of Her2CAR construction have been reported in our previous publication (Fu X, et al., A simple and sensitive method for measuring tumor-specific T cell cytotoxicity. PLoS One 2010; 5:e11867 (2010)). To measure cytokine release during CAR-mediated cytolysis, HUVEC or Her2-expressing 4T1-Her2 were mixed with the corresponding T-CARs at a 1:5 ratio in 48-well plates. As all the T-CARs were prepared from splenocytes obtained from C57BL/6 mice, they presented as allogeneic effector T cells for the 4T1-Her2 target. The culture supernatants were collected after 24 h incubation. The quantity of IL-2 and IFN-γ was determined by ELISA as per the manufacturer's instructions (R&D Systems, Minneapolis, Minn.).

Animal experiments. For establishing tumors, 1×10⁵ B16-F0 murine melanoma cells were implanted into the right flank of 6- to 8-week old male immunocompetent C57BL/6 mice (Taconic Farms, Hudson, N.Y.). When tumors became palpable (around day 5), mice were intravenously injected with either eCAR or SFG-GFP retrovirus-transduced splenocytes (4×10⁶ in 100 μl RPMI 1640) or PBS (n=10 mice per group). Tumor sizes were measured twice a week until the end of the experiment. Tumor volume was calculated by the following formula: tumor volume (mm³)=[length (mm)]×[width (mm)]²×0.52.

To determine the effect of retrovirus-transfected splenocytes on tumor blood vessels, mice bearing sizable B16-F0 tumors (approximately 8 mm in diameter) were intravenously injected with either eCAR or SFG-GFP retrovirus-transduced splenocytes (5×10⁶ in 100 μl RPMI 1640) or PBS (n=3 mice each group). Mice were humanely sacrificed 3 days later and their tumors excised. Tumors were fixed in 10% formalin for 24 h and then in 70% ethanol for another 24 h. This was followed by dehydration overnight in the Shandon Excelsior ES Tissue processor™ (Thermo Scientific, Waltham, Mass.). Successive 5 μm thick sections were cut and dehydrated in xylene and in decreasing ethanol concentrations (100% to 50%). Sections were then stained with hematoxylin and eosin for observation and micrograph under the microscope.

To investigate nanoparticle delivery following tumor blood vessel destruction, eCAR or SFG-GFP retrovirus-transduced splenocytes were intravenously injected into tumorbearing mice as described above. Forty-eight h later, mice received intravenous injection of DSPC/CHOL/mPEG2000-DSPE liposome nanoparticles (100 μm in size) labeled with Rhodamine DHPE (FormuMax Scientific, Inc. Palo Alto, Calif.), at a dose of 10 mg/kg diluted in 100 μl PBS. Twenty-four h after liposome injection, mice were sacrificed and tumors as well as major organs including lungs, kidneys and liver were collected. The collected tumors and organs were fixed in 4% paraformaldehyde at 4° C. for 24 h and then treated with 25% sucrose for another 24 h at 4° C. before they were embedded in OCT. Consecutive 5 μm thick cryo-sections were prepared for observation and micrographed under the fluorescence microscope (Olympus BX51). The intensity of rhodamine image was quantitated with MicroSuite™ FIVE software. Briefly, five areas were randomly clicked in each slide to obtain the reading of intensity value. A total of three slides (one from each animal) were subjected for quantification to obtain the mean value of each treatment group.

Statistical Analysis. All quantitative data are reported as mean+/−SD. Statistical analysis was made for multiple comparisons using analysis of variance and Student's t-test. P value <0.05 was considered to be statistically significant.

What is claimed is:

1. A compound for killing cancer cells comprising:
a T-cell engrafted with a chimeric antigen receptor (CAR), wherein the CAR comprises a targeting moiety that has a strong binding affinity to αᵥβ₃ integrin.

2. The compound of claim 1, wherein the targeting moiety is an echistatin polypeptide.

3. The compound of claim 2, wherein a peptide sequence in the echistatin polypeptide is linked to the T cell zeta chain.

4. The compound of claim 1, wherein the targeting moiety is a mutated echistatin polypeptide that has a reduced binding affinity to α₅β₁ integrin.

5. The compound of claim 4, wherein the mutated echistatin polypeptide has a substitution of leucine for amino acid 28 of an endogenous echistatin polypeptide.

6. A method for engrafting T cells with a chimeric antigen receptor (CAR) comprising:
transducing T cells with a retroviral vector or lentiviral vector, the retro- or lentiviral vector comprising coding sequences for a T cell zeta chain and a targeting moiety that has a strong binding affinity to αᵥβ₃ integrin.

7. The method of claim 6, wherein the targeting moiety is an echistatin polypeptide.

8. The method of claim 6, wherein the targeting moiety is a mutated echistatin polypeptide that has a reduced binding affinity to α₅β₁ integrin.

9. The method of claim 8, wherein the mutated echistatin polypeptide has a substitution of leucine for amino acid 28 of an endogenous echistatin polypeptide.

10. The method of claim 6, wherein the retroviral or lentiviral vector further comprises a signal peptide.

11. The method of claim 7, wherein the retroviral or lentiviral vector further comprises a CD28 domain between the coding sequences for the T cell zeta chain and the echistatin polypeptide.

12. The method of claim 11, wherein the retroviral or lentiviral vector further comprises a c-Myc tag between the CD28 domain and the coding sequence for the echistatin polypeptide.

13. A method of killing cancer cells in a host comprising:
administering to the host T cells transduced with a chimeric antigen receptor (CAR), the CAR comprising a targeting moiety that has a strong binding affinity to αᵥβ₃ integrin.

14. The method of claim 13, wherein the targeting moiety is an echistatin polypeptide.

15. The method of claim 14, wherein a peptide sequence in the echistatin polypeptide is linked to the T cell zeta chain.

16. The method of claim 13, wherein the targeting moiety is a mutated echistatin polypeptide that has a reduced binding affinity to α₅β₁ integrin.

17. The method of claim 16, wherein the mutated echistatin polypeptide has a substitution of leucine for amino acid 28 of an endogenous echistatin polypeptide.

18. The method of claim 13, wherein the transduced T cells are co-administered with one or more antineoplastic small molecules.

19. The method of claim 13, wherein the transduced T cells are co-administered with one or more antiangiogenic agents.

20. The method of claim 13, wherein the antiangiogenic agents include at least one of angiopoietin 2, angiostatin, endostatin, platelet factor-4, avastin, aflibercept, sorafenib, sunitinib, pazopanib, vandetanib, vatalanib, cediranib, and axitinib.


SEQUENCE LISTING (3 sequences)

SEQ ID NO: 1 - protein, 49 aa, Echis carinatus
ECESGPCCRNCKFLKEGTICKRARGDDMDDYCNGKTCDCPRNPHKGPAT

SEQ ID NO: 2 - protein, 49 aa, Artificial Sequence
  synthetic
ECESGPCCRNCKFLKEGTICKRARGDDLDDYCNGKTCDCPRNPHKGPAT

SEQ ID NO: 3 - DNA, 7219 bp, Artificial Sequence
  synthetic
gatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggactggatctggcgcatcctcttcctcgtcggcgctgctaccggcgctcattctgaagctttcatggaatgcgaatcaggtccatgctgtcgtaactgcaagttccttaaggaaggtaccatctgtaagcgcgcacgtggtgatgatctcgacgactactgcaacggtaagacctgtgactgcccgagaaacccacacaagggtccagctaccctcgaggaacagaaactgatctctgaagaagacctggtcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccagctcgaacgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagtt